(19) United States
(12) Patent Application Publication
(10) Pub. No.: US 2016/0081614 A1
(43) Pub. Date: Mar. 24, 2016

(54) EXERCISE ANALYSIS DEVICE, EXERCISE ANALYSIS METHOD, AND STORAGE MEDIUM HAVING EXERCISE ANALYSIS PROGRAM

(71) Applicant: CASIO COMPUTER CO., LTD., Tokyo (JP)

(72) Inventor: Takehiro Aibara, Tokyo (JP)

(21) Appl. No.: 14/858,837

(22) Filed: Sep. 18, 2015

(30) Foreign Application Priority Data
Sep. 22, 2014 (JP) 2014-193120

(51) Int. Cl. A61B 5/00; A61B 5/11

(52) U.S. Cl. CPC A61B 5/486; A61B 5/1118; A61B 5/7278; A61B 5/1112; A61B 5/742; A61B 2562/0219

(57) ABSTRACT

An exercise analysis device includes an acceleration acquiring unit, a first-data acquiring unit, a second-data acquiring unit and a third-data acquiring unit. The acceleration acquiring unit acquires acceleration of a user as the user performs an exercise. The first-data acquiring unit acquires a piece of a first data corresponding to a total amount of mechanical work of the exercise for a predetermined time based on the acceleration. The second-data acquiring unit acquires a piece of a second data corresponding to one of velocity and kinetic energy in a certain direction among directions related to the exercise for the predetermined time, based on the acceleration. The third-data acquiring unit acquires a piece of a third data corresponding to efficiency of the exercise based on the piece of the first data and the piece of the second data.

### CROSS-REFERENCE TO RELATED APPLICATION

The present application claims the priority of Japanese Patent Application No. 2014-193120 filed on Sep. 22, 2014, the contents of which being here incorporated for reference.

### FIELD OF THE INVENTION

The present invention relates to an exercise analysis device, an exercise analysis method, and a storage medium having an exercise analysis program stored thereon.

### DESCRIPTION OF THE RELATED ART

Marathon is booming so much that large-scale civic marathons are newly held in big cities. Besides, because of rising health consciousness, more and more people are performing daily exercise, such as running, walking, and cycling, to maintain their wellness or improve their health conditions. In addition, an increasing number of people are aiming to participate in sports games such as a marathon through their daily exercise. These people are very conscious of and interested in measuring and recording numerical values and data representing various biological information and exercise information so as to grasp their own health conditions and exercise statuses. Also, the people aiming to participate in sports games have an objective of achieving a successful record in those sports games, and therefore are very conscious of and interested in efficient and effective training methods.

In order to fulfill these demands, various products and technologies for runners have been developed as of now. For example, in JP-A-2010-264246, there is disclosed a portable fitness monitoring device which provides various biological information and exercise information to a user while the user is training. If the user wears various sensors such as a heart rate meter, an accelerometer, and a GPS receiver in order to use the portable fitness monitoring device, in the user exercise, the portable fitness monitoring device measures various performance parameters such as heart rate, distance, velocity, the number of steps, and calorie consumption, and provides the performance parameters as current information to the user.

Also, for example, in JP-A-2006-110046, there is disclosed a running-style learning device which a track and field athlete uses to practice running. When the user runs, the running-style learning device detects acceleration and angular velocity of each of three axis directions, and provides the results of comparison of the detected values with pre-set target values, and prompts the user to check and correct the running style, step by step.

### BRIEF SUMMARY OF THE INVENTION

However, most of people steadily who exercise steadily to maintain their wellness, including people aiming to participate in sports games or the like, have very few opportunities to be appropriately coached by an instructor or the like with regard to their exercise methods, their exercise form, etc. Also, it is very difficult for those people to grasp their body balance at the time of exercise (for example, running) and judge whether their body balance is appropriate. However, in a case where people continue exercise with their bodies out of balance, there are problems that the exercise is not only inefficient but also may damage their bodies.

With respect to this, the above-described devices and technologies just detect the biological information and exercise information when a user exercises and provide the detected information or the analysis results thereof to the user, and do not provide information on the form of the user, how to use the user's body, and the like in the user exercise.

Meanwhile, as devices for measuring the form of a user at the time of exercise such as running, for example, imaging devices for shooting a video or a high-speed video are on sale at relatively low prices. However, these imaging devices have problems that when a user exercises, in order to shoot a video, cooperation of someone other than the user is required, and it is impossible to feed the shooting result, the analysis result thereof, and the like back to the user who is exercising, in real time.

Also, as for image analysis or analytic diagnosis of exercise forms and the like, in general, large-scale, complex, and expensive apparatuses are required. Therefore, only in some of educational organizations, sports associations, and the like, it is possible to measure exercise forms and the like. For this reason, there are problems that it is difficult to measure exercise forms and the like of people who daily practice in places such as roads, parks, and playgrounds, and it is difficult for ordinary people other than top-level athletes to use those apparatuses.

An exercise analysis device of the present invention includes an acceleration acquiring unit, a first-data acquiring unit, a second-data acquiring unit and a third-data acquiring unit. The acceleration acquiring unit acquires acceleration of a user as the user performs an exercise. The first-data acquiring unit acquires a piece of a first data corresponding to a total amount of mechanical work of the exercise for a predetermined time, based on the acceleration. The second-data acquiring unit acquires a piece of a second data corresponding to one of velocity and kinetic energy in a certain direction among directions related to the exercise for the predetermined time, based on the acceleration. The third-data acquiring unit acquires a piece of a third data corresponding to efficiency of the exercise based on the piece of the first data and the piece of the second data.

An exercise analysis method includes: acquiring acceleration of a user as the user performs an exercise: acquiring a piece of a first data corresponding to a total amount of mechanical work of the exercise for a predetermined time, based on the acceleration; acquiring a piece of a second data corresponding to one of velocity and kinetic energy in a certain direction among directions related to the exercise for the predetermined time, based on the acceleration; and acquiring a piece of a third data corresponding to efficiency of the exercise based on the piece of the first data and the piece of the second data.

A non-transitory computer-readable storage medium has a program executable by a computer which comprises a controller. The program controls the controller to perform functions including: acquiring acceleration of a user as the user performs an exercise; acquiring a piece of a first data corresponding to a total amount of mechanical work of the exercise for a predetermined time, based on the acceleration; acquiring a piece of a second data corresponding to one of velocity and kinetic energy in a certain direction among directions related to the exercise for the predetermined time, based on the acceleration; and acquiring a piece of a third data corresponding to efficiency of the exercise based on the piece of the first data and the piece of the second data.

### BRIEF DESCRIPTION OF THE DRAWINGS

If the following detailed description is considered in conjunction with the accompanying drawings, deeper understanding of the present invention can be obtained.

FIGS. 1A to 1C are views illustrating the external appearance of an exercise analysis device according to an embodiment of the present invention.

FIGS. 2A and 2B are block diagrams illustrating an example of the hardware configuration of the exercise analysis device according to the embodiment.

FIG. 3 is a flow chart illustrating an example of an exercise analysis process according to the embodiment.

FIG. 4 is an explanatory view illustrating three axis directions of a gyro sensor 201 and an acceleration sensor 202 which are applied to the embodiment.

FIGS. 5A and 5B are explanatory views of an axis estimation process.

FIG. 6 is an explanatory view of a cycle estimation process.

FIGS. 7A and 7B are views illustrating examples of outputs of the acceleration sensor and the gyro sensor corresponding to one cycle.

FIG. 8 is a view illustrating an image of acceleration which occurs at the waist of a runner during running.

FIGS. 9A and 9B are explanatory views of a method of calculating a total of acceleration in an integration process.

FIG. 10 is a view illustrating a waveform data example of a front-rear direction acceleration component.

FIGS. 11A and 11B are views illustrating display examples of a display unit.

FIG. 12 is a view illustrating another display example of the display unit.

### DETAILED DESCRIPTION OF THE INVENTION

Hereinafter, an embodiment of the present invention will be described in detail with reference to the accompanying drawings. The present invention is an invention related to acquiring of data and processing of the acquired data when a runner wearing a sensor terminal runs.

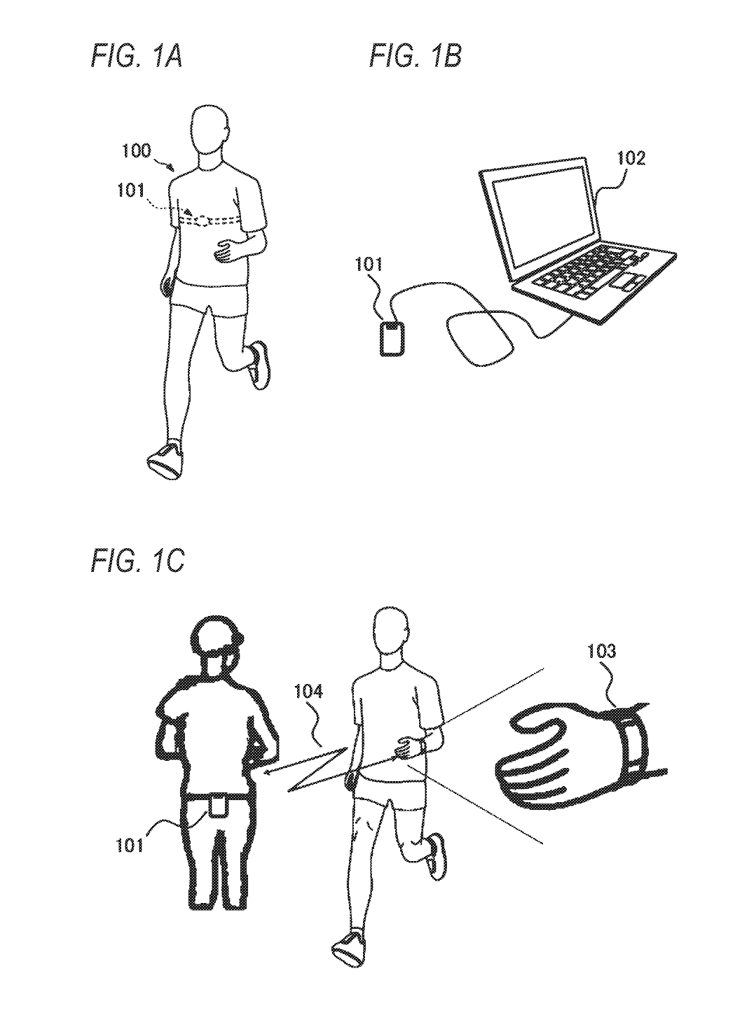

FIG. 1 shows an example in which a runner is wearing a sensor terminal 101. A runner (a user) 100 wears the sensor terminal 101 such that the sensor terminal is positioned on the chest as shown in FIG. 1A or on the back of the waist as shown in FIG. 1C. However, the runner may wear the sensor terminal 101 on any other body part such as the back of the neck such that the sensor terminal is positioned at a location which is the same distance from both ends in a left-right direction, that is, a location on a center line in a left-right direction when the runner is seen in a plan view.

FIGS. 1B and 1C are views illustrating examples of an analysis-result outputting method. FIG. 1B shows a combination in which after running finishes, data acquired by the sensor terminal 101 is transmitted to and displayed on a personal computer 102. FIG. 1C shows a combination in which, during running, in real time, the sensor terminal 101 acquires data, and analyzes the data, and wirelessly transmits the analysis result to a portable display unit 103 such as a wristwatch such that the display unit displays the analysis result.

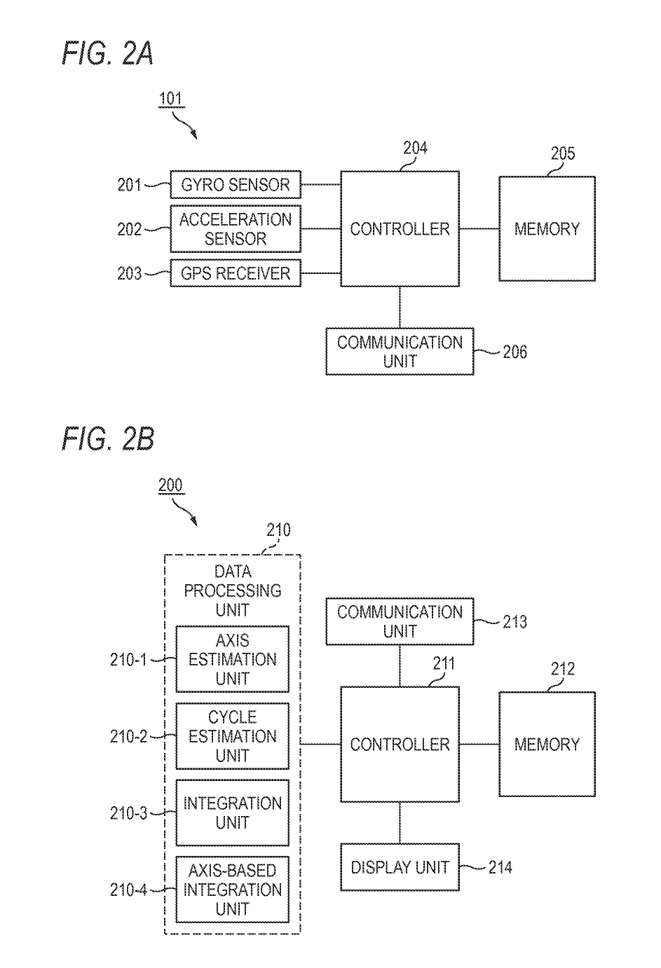

FIGS. 2A and 2B are block diagrams illustrating an example of the hardware configuration of the exercise analysis device according to the present embodiment. FIG. 2A shows an example of the hardware configuration of the sensor terminal 101, and FIG. 2b shows an example of the hardware configuration of the personal computer 102 of FIG. 1B or a data analyzing terminal 200 corresponding to the display unit 103 of FIG. 1C.

In FIG. 2A, the sensor terminal 101 includes a gyro sensor 201, an acceleration sensor 202, a GPS (global position system) receiver 203, a controller 204, a memory 205, and a communication unit 206.

The gyro sensor 201 detects angular velocity in the revolving direction of revolving exercise on the measurement axis of the gyro sensor (in the present embodiment, this measurement axis is substantially parallel to a body axis of the runner 100 (FIG. 1)). Alternatively, in place of the gyro sensor 201, any other means capable of detecting angular velocity can be used.

The acceleration sensor 202 detects acceleration of each of three directions which are extensions of the measurement axes of the acceleration sensor (in the present embodiment, these measurement axes are substantially parallel to body axes of the runner 100). Alternatively, any other means capable of detecting acceleration can be used.

The GPS receiver 203 detects velocity data and information on the location of the runner 100. Alternatively, any other means capable of detecting velocity data can be used.

The controller 204 acquires output data of each of the gyro sensor 201, the acceleration sensor 202, and the GPS receiver 203, and saves the acquired data in the memory 205. Also, the controller 204 transmits the data saved in the memory 205, to the data analyzing terminal 200 through the communication unit 206.

In FIG. 2B, the data analyzing terminal 200 includes a data processing unit 210, a controller 211 (a third-data acquiring unit), a memory 212, a communication unit 213, and a display unit 214. The data processing unit 210 is, for example, a digital signal processor (DSP), and includes an axis estimation unit 210-1, a cycle estimation unit 210-2, an integration unit 203-3 (a first-data acquiring unit), and an axis-based integration unit 210-4 (a second-data acquiring unit). Details of these units will be described below.

The controller 211 receives data from the sensor terminal 101 of FIG. 2A through the communication unit 213, and transmits the received data to the data processing unit 210, and saves intermediate data or result data obtained by calculations in the data processing unit 210, in the memory 212.

In the physical education field, there has been proposed various indexes EI for representing the efficiency of running, and an index EI which is most frequently used is expressed as the following Expression 1.

$$EI = \frac{\begin{matrix}KINETIC ENERGY INPROPULSION \\ DIRECTION FOR ONE EXERCISE CYCLE\end{matrix}}{\begin{matrix}TOTAL AMOUNT OF WORK OF WHOLE \\ BODY FOR ONE EXERCISE CYCLE\end{matrix}} \qquad [ Expression 1 ]$$

Expression 1 represents general energy efficiency, and in the numerator, kinetic energy in the propulsion direction (running direction) of the runner 100 is placed as effective energy, and in the denominator, the total amount of work which the whole body of the runner 100 has done is placed. In the present embodiment, the propulsion direction of the runner 100 is parallel to a horizontal plane. That is, the index which is expressed as Expression 1 represents how much work which has been done by the whole body has contributed to moving velocity in a horizontal direction.

According to the present embodiment, the total amount of work of the whole body of a runner which it is very difficult to measure is replaced with a total of acceleration of the torso of the runner having the largest mass in the whole body, whereby many people can evaluate the efficiency of running with simple devices even though the evaluated efficiency is not efficiency in the strict sense.

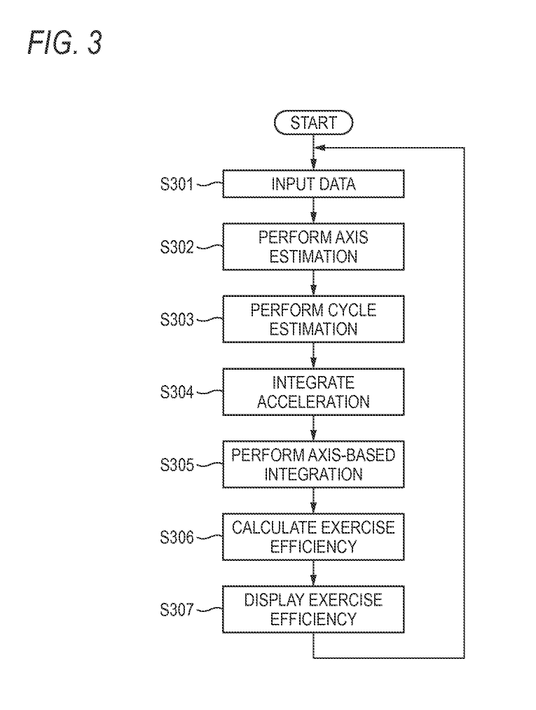

FIG. 3 is a flow chart illustrating an example of an exercise analysis process which is related to the present embodiment and is performed by the data analyzing terminal 200 having the hardware configuration shown as an example in FIG. 2B. This process is implemented as digital signal processing of the data analyzing terminal 200, and processing in which the controller 211 executes the exercise analysis program stored in the memory 212.

First, in STEP S301 of FIG. 3, the controller 211 receives data of each of an output of the gyro sensor 201, an output of the acceleration sensor 202, and an output of the GPS receiver 203, from the sensor terminal 101 through the communication units 206 and 213, and transmits the received data to the data processing unit 210.

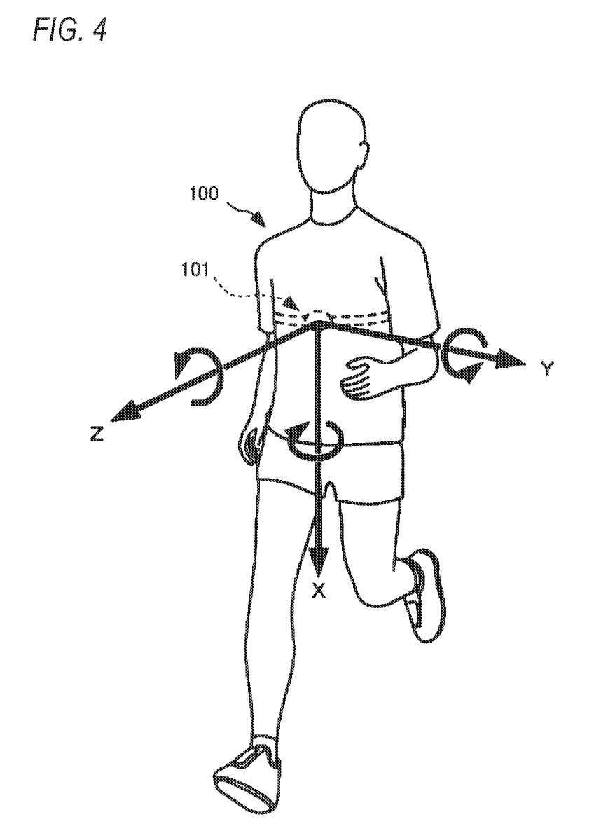

FIG. 4 is an explanatory view illustrating three axis directions of the gyro sensor 201 and the acceleration sensor 202 which are used in the present embodiment. In the present embodiment, the acceleration sensor 202 measures the rate of change of the motion velocity (acceleration) when the runner 100 exercises. In the present embodiment, the acceleration sensor 202 has three axis acceleration sensors, and detects acceleration components along three axis directions perpendicular to one another, and outputs the acceleration components as acceleration data. That is, with respect to the runner 100, an axis extending in a vertical direction is referred to as an x axis, and an acceleration component directed downward (toward the ground) is defined as a positive component. Here, the x axis is substantially aligned with the extension direction of the body axis of the runner 100. Further, with respect to the runner 100, an axis extending in a left-right direction is referred to as a y axis, and an acceleration component directed toward the left hand is defined as a positive component. Furthermore, with respect to the runner 100, an axis extending in a front-rear direction is referred to as a z axis, and an acceleration component directed forward (toward the movement direction) is defined as a positive component. The acceleration data acquired by the acceleration sensor 202 is associated with time data which is generated by the controller 204, and is input to the controller 211. Therefore, the controller 211 acts as an acceleration acquiring unit which acquires acceleration when the runner 100 exercises.

The gyro sensor 201 measures change of the motion direction (angular velocity) when the runner 100 exercises. In the present embodiment, the gyro sensor 201 includes three axis angular-velocity sensors, and detects angular velocity components generated in the revolving direction of revolving exercise on each axis of three axes perpendicular to one another, and outputs those angular velocity components as angular velocity data. Here, as shown in FIG. 4, with respect to three axes perpendicular to one another, that is, the x, y, and z axes, an angular velocity component which is generated in the clockwise direction toward the positive side of each axis is defined as a positive angular velocity component. Here, an angular velocity component which is generated in the revolving direction on the x axis substantially coincides with angular acceleration which is generated on the body axis of the runner 100. The angular velocity data acquired by the gyro sensor 201 is associated with the time data which is generated by the controller 204, and is input to the controller 211. Therefore, the controller 211 acts as an acceleration acquiring unit which acquires angular acceleration when the runner 100 exercises.

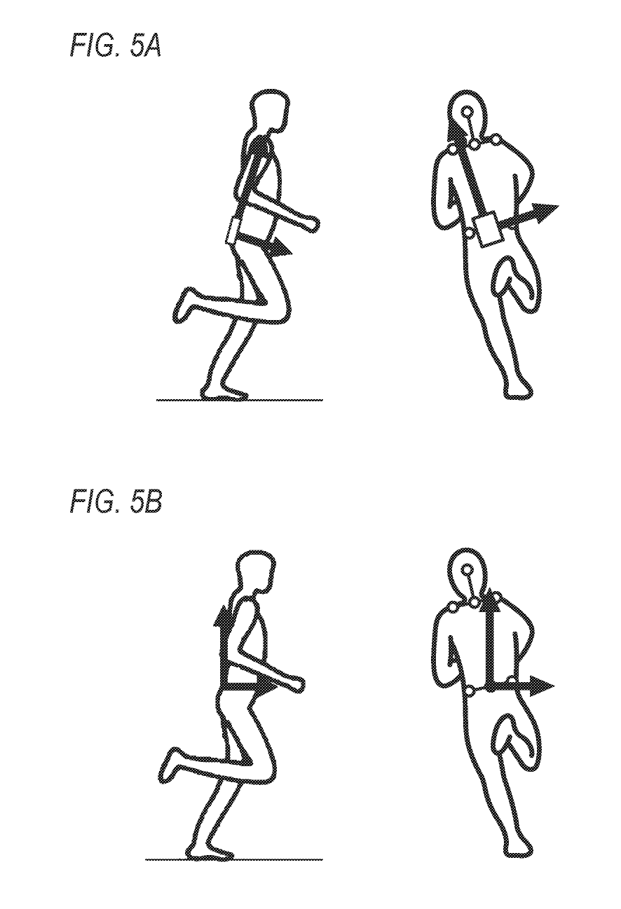

Subsequently, in STEP S302 of FIG. 3, the axis estimation unit 210-1 of the data processing unit 210 performs an axis estimation process. FIGS. 5A and 5B are explanatory views of the axis estimation process. For example, in a case where the sensor terminal 101 is positioned on the waist, when the runner 100 runs, the runner leans to either side while bending forward, as shown in FIG. 5A. The axis estimation process is a process of estimating the tilt based on the data of the acceleration sensor 202 and the gyro sensor 201 and converting the estimated tilt into data along an axis relative to a vertical direction, that is, axis coordinate data when a y axis and a z axis are taken along a horizontal direction and a vertical direction is defined as an x axis direction, as shown in FIG. 5B. As an example of that estimation scheme, for example, a three-axis output of the acceleration sensor 202 and a three-axis output of the gyro sensor 201 are input to a Kalman filter or a lowpass filter, whereby it is possible to calculate three-axis data on acceleration relative to the ground (a horizontal surface) and three-axis data on angular velocity. Also, in the present embodiment, besides a Kalman filter or a lowpass filter, any other axis estimation scheme can be used.

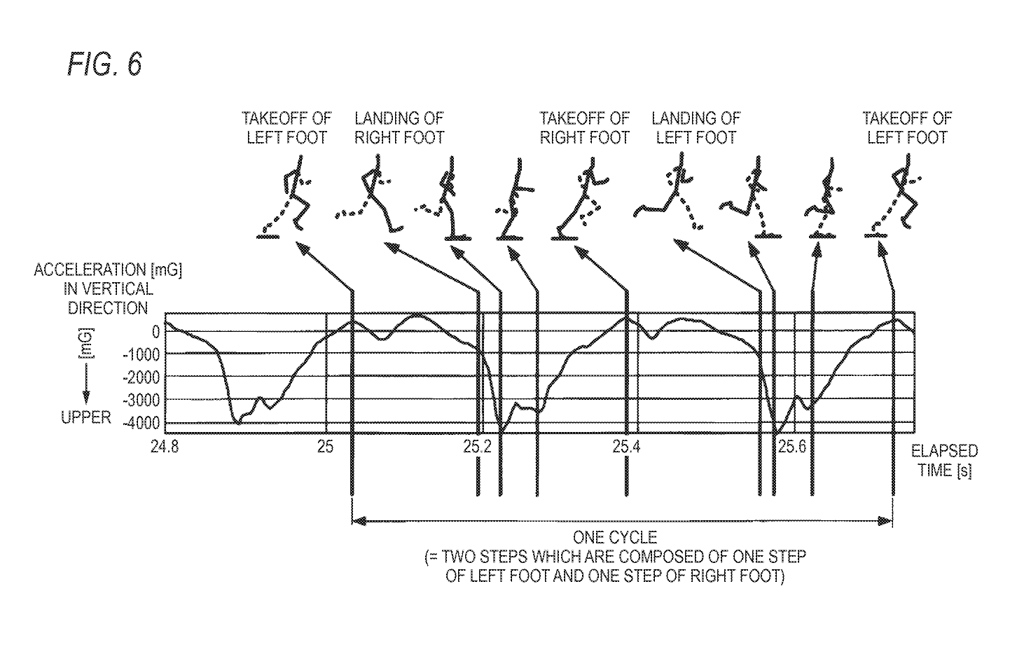

Subsequently, in STEP S303 of FIG. 3, the cycle estimation unit 210-2 of the data processing unit 210 performs a cycle estimation process. FIG. 6 is an explanatory view of the cycle estimation process. In general, in running motions of running or the like, for example, as shown in the upper portion of FIG. 6, a sequence of takeoff of one foot (in FIG. 6, takeoff of the left foot), landing and takeoff of the other foot (landing and takeoff of the right foot), and landing and takeoff of the one foot (landing and takeoff of the left foot), that is, total two steps of one step of the left foot and one step of the right foot can be defined as one cycle (one running cycle or one exercise cycle). Meanwhile, in the series of running motions, a vertical direction acceleration component of acceleration data acquired by the acceleration sensor 202 and corrected by the axis estimation unit 210-1 shows a signal waveform having periodicity for one step of the left foot or the right foot, for example, as shown in the lower portion of FIG. 6. For this reason, two cycles in the vertical direction acceleration component corresponds to one cycle in the running motions (one running cycle). Therefore, based on the vertical direction acceleration component acquired by the acceleration sensor 202 and corrected by the axis estimation unit 210-1, it is possible to stably segment motion data of each cycle (which is the cycle of a series of motions in which the left foot and the right foot alternately move once, and will be hereinafter referred to as the motion cycle) of running motions having been done by the runner 100. With this, it is possible to accurately measure the time of the corresponding one cycle. Therefore, the cycle estimation unit 210-2 acts as a time estimation unit which estimates a time when the runner 100 has done a predetermined motion having periodicity, as one cycle described above. Also, as the cycle estimation process, any other scheme can be used.

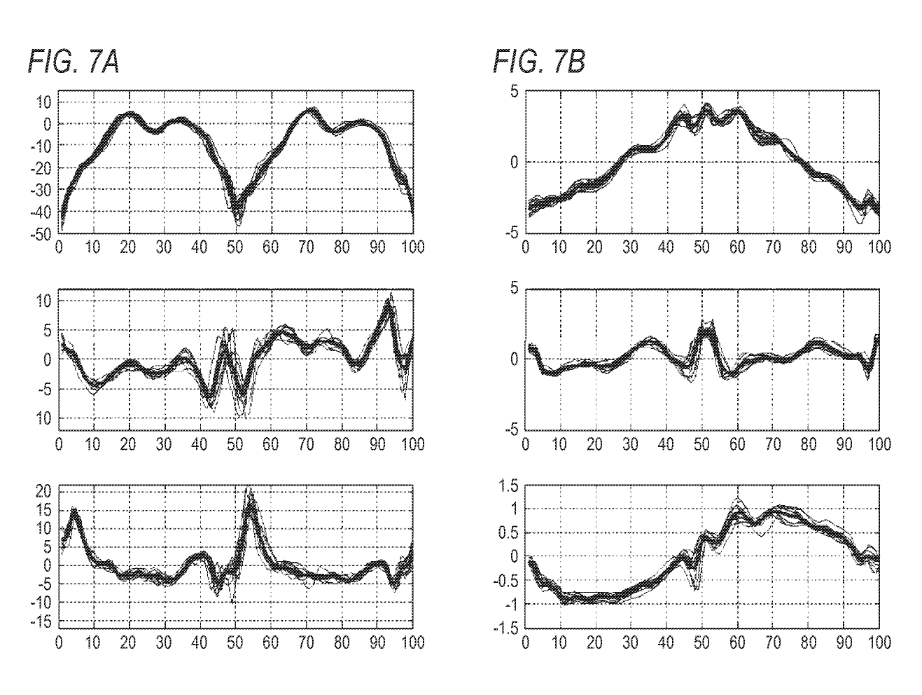

FIGS. 7A and 7B are views illustrating examples of outputs of acceleration data and angular velocity data of one cycle after the outputs of the acceleration sensor 202 and the gyro sensor 201 have been corrected by the axis estimation unit 210-1, respectively. In FIGS. 7A and 7B, from the top, data relative to the front-rear direction, the left-right direction, and the vertical direction are sequentially shown. The horizontal axis of each graph represents normalized amount which is obtained by normalizing times of the one cycle to 0 through 100, which requires no unit of quantity. The vertical axis of FIG. 7A represents acceleration, which unit is “m/s²”. The vertical axis of FIG. 7B represents angular velocity, which unit is “rad/s”.

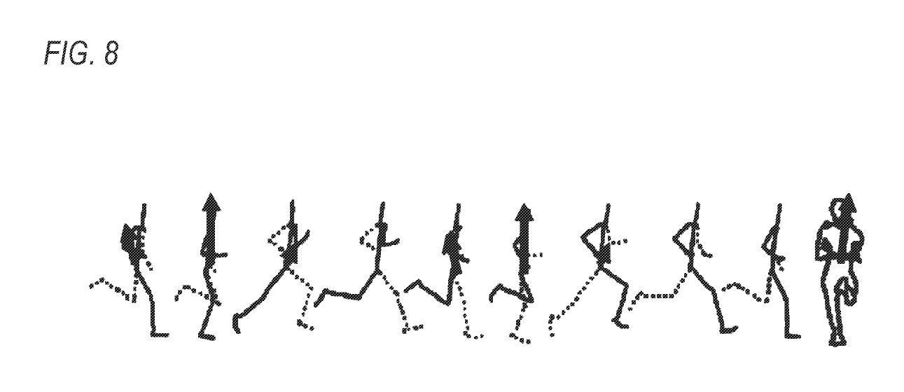

Subsequently, in STEP S304 of FIG. 3, the integration unit 203-3 of the data processing unit 210 performs an acceleration integration process. In this process, regardless of the direction of acceleration, the magnitude of acceleration generated at the waist part, that is, a part where the sensor terminal 101 is positioned is integrated for one cycle. Therefore, the integration unit 203-3 acts as the first-data acquiring unit which acquires data (first data) corresponding to the total amount of mechanical work based on exercise of the runner 100 for one cycle (a predetermined time) based on acceleration. FIG. 8 is a view illustrating an image of acceleration which is generated at the waist during running. While a foot is on the ground, acceleration close to a ground reaction force which the foot receives from the ground occurs at the waist, and in addition to that, acceleration based on the motion of the waist according to a running form occurs. Also, while both feet are floating in the air, acceleration based on the motion of the waist according to the running form occurs.

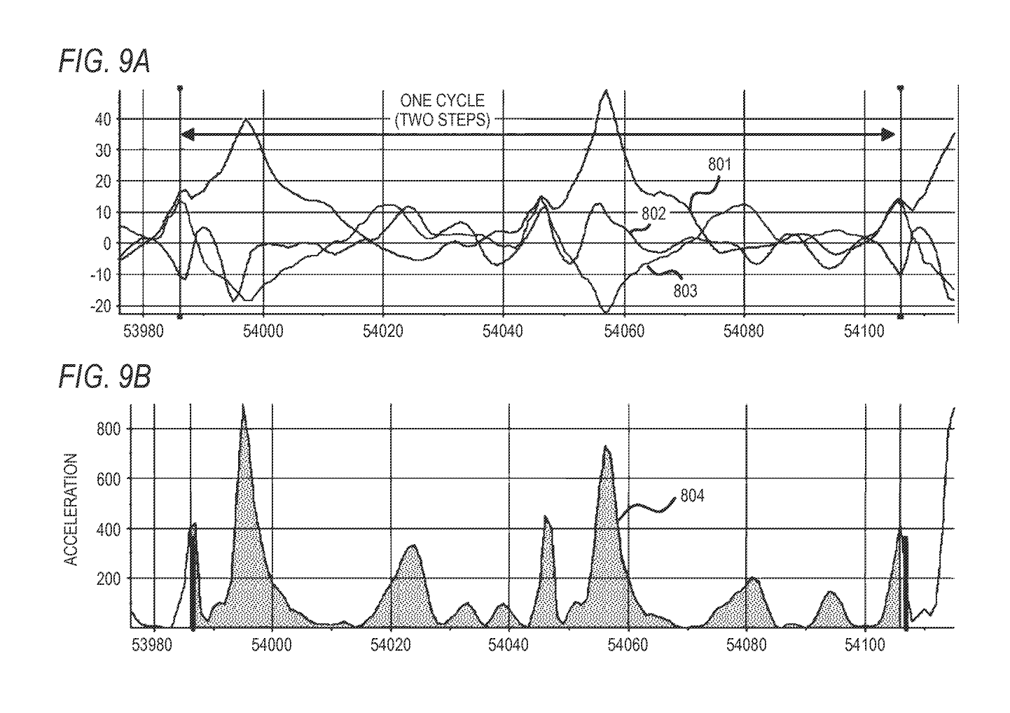

FIGS. 9A and 9B are explanatory views of a method of calculating a total of acceleration in the acceleration integration process. The horizontal axis of each graph represents period elapsing from the start time of measurement, which unit is “s”. The vertical axis of FIG. 9A represents acceleration, which unit is “m/s²”. The vertical axis of FIG. 9B represents square of acceleration, which unit is “m²/s⁴”. Data to be output by the acceleration sensor 202 is obtained as acceleration components relative to axes of three directions perpendicular to one another as described with reference to FIG. 4. Similarly, an output of the axis estimation unit 210-1 is also obtained as acceleration components relative to axes of three directions perpendicular to one another by correction as described with reference to FIGS. 5A and 5B. In FIG. 9A, a graph 801 represents a corrected vertical direction acceleration component Ax, and a graph 802 represents a corrected left-right direction acceleration component Ay, and a graph 803 represents a corrected front-rear direction acceleration component Az. In the present embodiment, in order to calculate the magnitude of acceleration at each moment, the root (square root) of the sum of squares of the individual components is calculated as the following Equation 2, whereby the magnitude “A” of acceleration shown in FIG. 9B is calculated.

A=\sqrt{(Ax)²+(Ay)²+(Az)²} [Expression 2]

Subsequently, the magnitude “A” of acceleration obtained at each moment is integrated for one cycle which is the exercise cycle calculated in the cycle estimation process of STEP S303 of FIG. 3, as shown in FIG. 9B, whereby the total of acceleration for one cycle which is the motion cycle is calculated. Therefore, the integration unit 203-3 acts as the first-data acquiring unit which acquires the above described first data by acquiring the magnitude of acceleration in each direction and integrating the corresponding acceleration magnitude for one cycle.

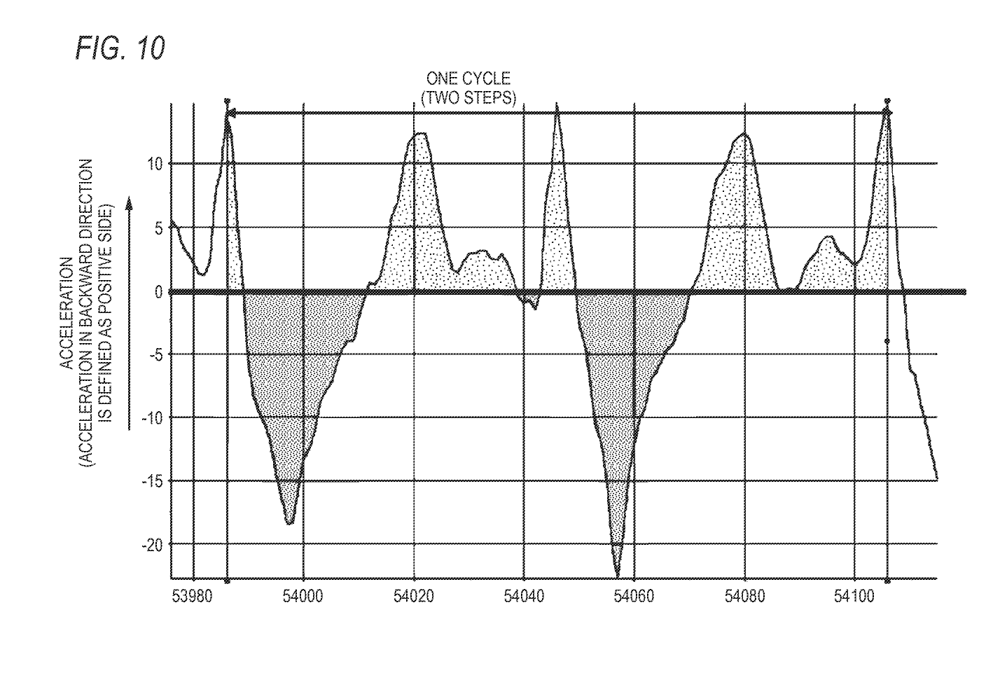

Subsequently, in STEP S305 of FIG. 3, the axis-based integration unit 210-4 of the data processing unit 210 performs an axis-based integration process. In this process, with respect to waveform data on the front-rear direction (horizontal direction) acceleration component Az which is one of the acceleration components of three directions obtained as shown in FIG. 9A and is represented by the graph 803, an integration process for one cycle which is the motion cycle is performed. Therefore, the axis-based integration unit 210-4 acts as the second-data acquiring unit which acquires data (the second data) corresponding to kinetic energy of a predetermined direction of directions related to exercise of the runner 100 for one cycle (a predetermined time), based on acceleration. FIG. 10 is a view illustrating a waveform data example of the front-rear direction acceleration component. The horizontal axis of this graph represents period elapsing from the start time of measurement, which unit is “s”. The vertical axis of this graph represents acceleration, which unit is “m/s²”. Backward (brake component) acceleration of the runner 100 becomes positive. A value obtained by integrating the absolute values of negative components of the backward acceleration components for one cycle which is the motion cycle becomes the sum of acceleration components of the propulsion direction of the runner 100 for one cycle which is the motion cycle. Also, since the running becomes uniform velocity exercise, if the value obtained by integrating the absolute values of the negative components for one cycle which is the motion cycle is subtracted from the value obtained by integrating the positive components of the backward acceleration components for one cycle which is the motion cycle, “0” is obtained. Therefore, the value obtained by integrating the positive components also becomes the sum of the acceleration components of the propulsion direction of the runner 100 for one cycle which is the motion cycle. Therefore, the axis-based integration unit 210-4 acts as the second-data acquiring unit which acquires the second data by acquiring acceleration of the propulsion direction of the runner 100 based on acceleration and angular velocity and integrating acceleration of any one side of the positive side and negative side of the propulsion direction of the corresponding runner 100 for one cycle.

Alternatively, easily, in order to calculate the magnitude of acceleration at each moment, a value “A” based on the magnitude of acceleration shown in FIG. 9B may be calculated by calculating the root of the sum of squares of components of two directions including at least the z direction component as the following Expression 3 or 4.

A=\sqrt{(Ax)²+(Az)²} [Expression 3]

A=\sqrt{(Ay)²+(Az)²} [Expression 4]

Even in this case, the value “A” based on the magnitude of acceleration obtained at each moment is integrated for one cycle which is the motion cycle, whereby the total of acceleration for one cycle which is the motion cycle is calculated. Therefore, the integration unit 203-3 acts as the first-data acquiring unit which acquires the above described first data by acquiring the magnitude of acceleration of each direction and integrating the corresponding acceleration magnitude for one cycle.

Further, regardless of the propulsion direction of the runner 100, in order to calculate the index of exercise efficiency relative to a direction perpendicular to the propulsion direction of the runner 100 in a horizontal plane, or a vertical direction, the root of the sum of squares of two direction components including at least the y direction component or the x direction component may be calculated. Therefore, the integration unit 203-3 acts as the first-data acquiring unit which acquires the above described first data by acquiring the magnitude of acceleration of each direction and integrating the corresponding acceleration magnitude for one cycle.

If the acceleration magnitude “A” calculated in the acceleration integration process of STEP S304 of FIG. 3 is used, the total amount W of mechanical work based on the running exercise of the runner 100 for one cycle which is the motion cycle is expressed as the following Expression 5. In Expression 5, the integral sign and dt represent integration for one cycle which is the motion cycle. Also, in Expression 5, “M” represents the weight of the runner 100.

W=M×∫Adt [Expression 5]

Meanwhile, if the acceleration magnitude Az calculated by the axis-based integration unit of STEP S305 of FIG. 3 is used, kinetic energy Wz of the propulsion direction for one cycle which is the motion cycle is expressed as the following Expression 6. Similarly in Expression 5, in Expression 6, the integral sign and dt represent integration for one cycle which is the motion cycle. Also, similarly in Expression 5, in Expression 6, “M” represents the weight of the runner 100.

Wz=M×∫Azdt [Expression 6]

Therefore, if Expression 5 and Expression 6 are assigned to the numerator and denominator of Expression 1, respectively, it is possible to calculate the efficiency of kinetic energy of the propulsion direction of the runner 100 relative to the total amount of mechanical work based on the running exercise of the runner 100 for one cycle which is the motion cycle as shown by the following Expression 7.

Wz/W=∫Azdt/∫Adt [Expression 7]

Also, if it is possible to detect the average running velocity (velocity of the propulsion direction) Vz of the runner 100 for one cycle which is the motion cycle, based on an output of the GPS receiver (a GPS sensor and a velocity acquiring unit) 203 of FIG. 2A, it is possible to calculate the kinetic energy of the propulsion direction of the runner 100 by the following Expression 8. Also, similarly in Expression 5 and the like, in Expression 8. “M” represents the weight of the runner 100. Therefore, the axis-based integration unit 210-4 acts as the second-data acquiring unit which acquires the above described second data by dividing the square of velocity detected by the GPS receiver 203 by 2. Therefore, the GPS receiver 203 acts as the velocity acquiring unit which acquires the velocity of the propulsion direction of the runner 100 based on the output of the GPS sensor.

Wz=½MVz² [Expression 8]

Therefore, if Expression 8 and Expression 5 are assigned to the numerator and denominator of Expression 1, respectively, it is possible to calculate the efficiency of kinetic energy of the propulsion direction of the runner 100 relative to the total amount of mechanical work based on the running exercise of the runner 100 for one cycle which is the motion cycle as shown by the following Expression 9. Therefore, the controller 211 acts as the third-data acquiring unit which acquires the ratio of the first data and the second data described above, that is, the efficiency of kinetic energy of any one side of the positive side and negative side of the propulsion direction of the runner 100 relative to the total amount of mechanical work based on the exercise of the runner 100 for one cycle, as third data.

Wz/W=½Vz²/∫Adt [Expression 9]

Further, more easily, Expression 5 may be assigned to the numerator and be divided by velocity Vz, as shown by the following Expression 10, whereby the index can be calculated. Therefore, the axis-based integration unit 210-4 acts as the second-data acquiring unit which acquires velocity detected by the GPS receiver 203, as the above described second data.

W/Vz=∫Adt/Vz [Expression 10]

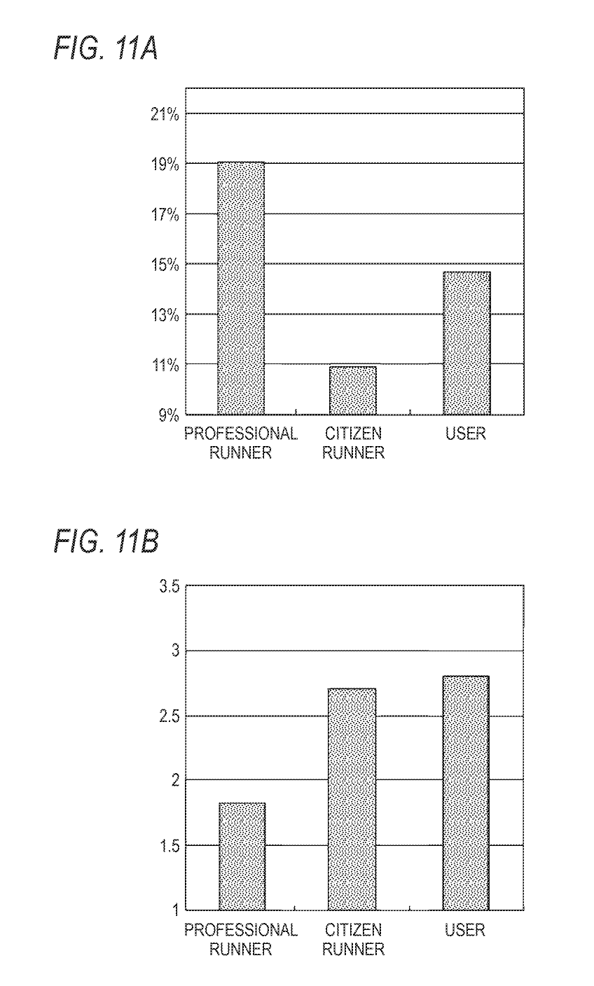

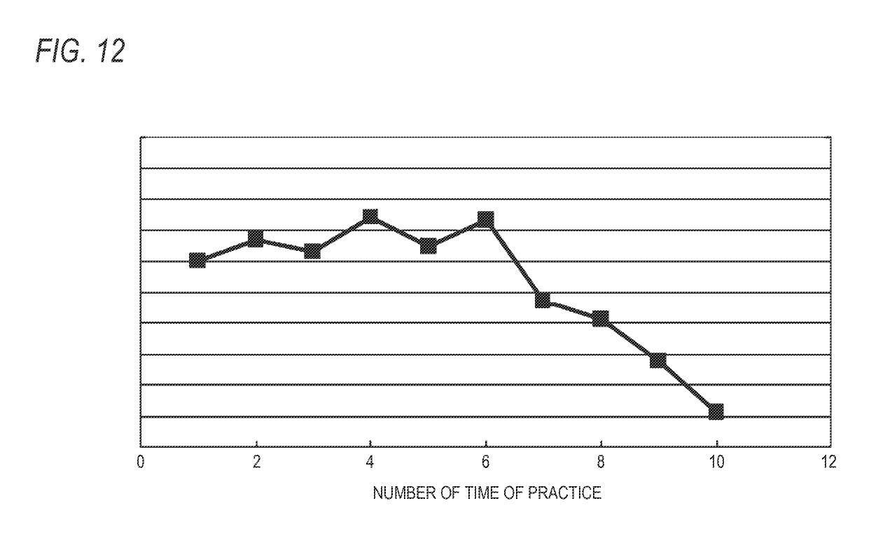

Returning to the flow chart of FIG. 3, the controller 211 of FIG. 2B calculates the index of exercise efficiency based on the above described Expression 7, 9, or 10, in STEP S306 of FIG. 3, and displays the calculated index on the display unit 214 of FIG. 2B in STEP S307 of FIG. 3. FIGS. 11A, 11B, and 12 are views illustrating display examples of the display unit 214. The horizontal axis of each graph represents the number of times of practice, which unit is “times”. Further, the vertical axis of each graph represents the index value of exercise efficiency, which requires no unit of quantity.

Therefore, the controller 211 acts as the third-data acquiring unit which acquires third data corresponding to the efficiency of exercise of the runner based on the first data and the second data described above.

FIG. 11A is a graph illustrating a comparison of the index value of the efficiency of current running of the runner with the index value of the efficiency of running of a professional runner and the index value of the efficiency of running of a citizen runner obtained as model data. In FIG. 11A, the vertical axis of the graph represents the index value of exercise efficiency which is calculated by “Wz/W=(TOTAL MAGNITUDE OF ACCELERATION OF PROPULSION DIRECTION FOR ONE CYCLE)/(TOTAL MAGNITUDE OF ACCELERATION OF ALL DIRECTIONS FOR ONE CYCLE)” according to Expression 7. Referring to FIG. 11A, in the case of the professional runner, the ratio of the total magnitude of the propulsion direction to the total magnitude of all directions is 19%; whereas in the case of the citizen runner, the corresponding ratio is less than 11%. The runner 100 can visually check where the runner is positioned at that moment.

FIG. 11B is a graph illustrating a comparison of the index value of the efficiency of current running of the runner with the index value of the efficiency of running of a professional runner and the index value of the efficiency of running of a citizen runner obtained as model data. In FIG. 11B, the vertical axis of the graph represents the index value of exercise efficiency which is calculated by “W/Vz=(TOTAL MAGNITUDE OF ACCELERATION OF ALL DIRECTIONS FOR ONE CYCLE)/(AVERAGE RUNNING VELOCITY FOR ONE CYCLE)” according to Expression 10. Therefore, acceleration of the waist per velocity can be seen, and as the index value decreases, the motion of the waist decreases and higher velocity is obtained. It can be seen that as compared to the model data, the index value of the runner shown on the rightmost side of FIG. 11B is worse than that of the professional runner but is not so bad among citizen runners. Further, it becomes possible to conduct research on a running manner to reduce the index value. Although the above described data is obtained by (TOTAL MAGNITUDE OF ACCELERATION)/(RUNNING VELOCITY), since the total magnitude is an integral value of acceleration for one cycle, if the data is normalized by (TOTAL MAGNITUDE OF ACCELERATION)/(CYCLE (TIME)), it is possible to compare the data with the lengths of cycle times excluded.

Referring to FIGS. 11A and 11B together, it can be seen that acceleration necessary for the professional runner to achieve velocity is small, and in this small acceleration, the ratio of acceleration which is used in the whole body is large. That is, it can be seen that the professional runner effectively uses small force to run in the propulsion direction. In other words, the citizen runner does many useless motions which do not contribute in the propulsion direction.

FIG. 12 is a graph illustrating change in the index value of exercise efficiency shown in FIG. 11A and calculated for every practice by “Wz/W=(TOTAL MAGNITUDE OF ACCELERATION OF PROPULSION DIRECTION FOR ONE CYCLE)/(TOTAL MAGNITUDE OF ACCELERATION OF ALL DIRECTIONS FOR ONE CYCLE)”. Referring to FIG. 12, it is possible to confirm that the efficiency becomes higher as the number of times of practice increases.

In the axis-based integration process of STEP S305 of FIG. 3, among the acceleration components of three directions shown in FIG. 9A, with respect to only the front-rear direction acceleration component Az shown by the graph 803, the result of integration for one cycle which is the motion cycle was obtained, and was used in display on the display unit 214. With respect to this, it is also possible to obtain the result of integration for one cycle which is the motion cycle with respect to even the vertical direction acceleration component Ax shown by the graph 801 in FIG. 9A or the left-right direction acceleration component Ay shown by the graph 802, and display the obtained result on the display unit 214 for comparison with the integration result of the acceleration components of all directions. In this case, it is possible to check how much the body is moving in the vertical direction and the left-right direction during running. Therefore, the axis-based integration unit 210-4 acts as the second-data acquiring unit which acquires data corresponding to kinetic energy of any one direction of the propulsion direction of the runner 100, a direction perpendicular to the propulsion direction of the runner 100 in a horizontal plane, and a vertical direction, as the above described second data.

Further, in the exercise efficiency calculating process of STEP S306 of FIG. 3, the running velocity Vz of the runner 100 detected based on the output of the GPS receiver 203 is used to calculate the index of exercise efficiency. However, it is also possible to obtain the average running velocity Vx or Vy of the runner 100 for one cycle which is the motion cycle with respect to even the acceleration component of the x axis direction or the y axis direction of FIG. 4, and display the obtained velocity on the display unit 214 for comparison with the integration result of the acceleration components of all directions. In this case, it is possible to easily check how much the runner is moving in the vertical direction and the left-right direction during running. Therefore, the axis-based integration unit 210-4 acts as the second-data acquiring unit which acquires data corresponding to velocity of any one direction of the propulsion direction of the runner 100, a direction perpendicular to the propulsion direction of the runner 100 in a horizontal plane, and a vertical direction, as the above described second data.

Returning to the flow chart of FIG. 3, after STEP S307, the control returns to STEP S301. Also, the exercise analysis process shown in FIG. 3 may be performed after exercise such as running finishes, or may be performed in real time during exercise. Especially, in the case of performing the exercise analysis process in real time, the runner can check display of exercise efficiency by himself while exercising, and correct his form in real time.

As described above, although a large-scale apparatus has been required for exercise analysis in the related art, according to the present embodiment, in place of the total amount of work of the whole body of a runner which it is very difficult to measure, a total of acceleration of the torso of the runner having the largest mass in the whole body is used, for example, in dividing the square of velocity or a total of acceleration of the propulsion direction by a total of acceleration of all directions. As a result, it becomes possible to analyze exercise with simple devices, and, for example, it is possible to see acceleration components of the whole acceleration which are used in the propulsion direction, the vertical direction, and the left-right direction, and it becomes possible to provide unprecedented new indexes of exercise efficiency even though the efficiency is not efficiency in the strict sense.

If the runner can see those indexes, the runner can determine the directivity of his practice and a plan.

Further, it also becomes possible to check practice effect about whether practice works.

Although some embodiments of the present invention have been described, the scope of the present invention is not limited to the above described embodiments, and includes the scopes of inventions disclosed in claims and the scopes of their equivalents.

The following is the inventions disclosed in the claims originally attached to this application. The numbering of the claims appended is the same as the numbering of the claims originally attached to this application.

What is claimed is:

1. An exercise analysis device comprising:
an acceleration acquiring unit that acquires acceleration of a user as the user performs an exercise;
a first-data acquiring unit that acquires a piece of a first data corresponding to a total amount of mechanical work of the exercise for a predetermined time, based on the acceleration;
a second-data acquiring unit that acquires a piece of a second data corresponding to one of velocity and kinetic energy in a certain direction among directions related to the exercise for the predetermined time, based on the acceleration; and
a third-data acquiring unit that acquires a piece of a third data corresponding to efficiency of the exercise based on the piece of the first data and the piece of the second data.

2. The exercise analysis device according to claim 1, wherein:
the first-data acquiring unit acquires a plurality of pieces of magnitude of acceleration in different directions, and integrates the plurality of pieces of magnitude of acceleration in the different directions for the predetermined time so as to acquire the piece of the first data.

3. The exercise analysis device according to claim 1, further comprising:
an angular velocity acquiring unit that acquires angular velocity in a revolving direction along a body axis of the user in the exercise,
wherein, based on the acceleration and the angular velocity, the second-data acquiring unit acquires acceleration in the certain direction, and integrates the acceleration in one of a positive side and a negative side of the certain direction for the predetermined time, so as to acquire the piece of the second data.

4. The exercise analysis device according to claim 3, wherein:
the second-data acquiring unit acquires, as the piece of the second data, data corresponding to kinetic energy of one of a traveling direction in the exercise, a direction perpendicular to the traveling direction in a horizontal plane, and a vertical direction.

5. The exercise analysis device according to claim 1, wherein:
the second-data acquiring unit acquires the piece of the second data by dividing the square of the velocity in the certain direction by 2.

6. The exercise analysis device according to claim 5, wherein:
the second-data acquiring unit acquires the piece of the second data based on an output of a GPS sensor.

7. The exercise analysis device according to claim 1, wherein:
the third-data acquiring unit acquires, as the third data, a ratio between the piece of the first data and the piece of the second data to obtain efficiency of one of the velocity and the kinetic energy in one of a positive side and a negative side of a traveling direction in the exercise with respect to the total amount of mechanical work, for the predetermined time.

8. The exercise analysis device according to claim 1, further comprising:
a display unit that displays information about the efficiency in the exercise, based on the third data,
wherein the display unit displays the piece of the third data of the user in the exercise and the piece of the third data of another user.

9. The exercise analysis device according to claim 1, wherein:
the predetermined time, when the user in the exercise performs a periodical motion, equals to time for each of the periodical motion, and
the exercise analysis device further comprises a time estimation unit that estimates a cycle of the periodical motion as the predetermined time.

10. An exercise analysis method comprising:
acquiring acceleration of a user as the user performs an exercise;
acquiring a piece of a first data corresponding to a total amount of mechanical work of the exercise for a predetermined time, based on the acceleration;
acquiring a piece of a second data corresponding to one of velocity and kinetic energy in a certain direction among directions related to the exercise for the predetermined time, based on the acceleration; and
acquiring a piece of a third data corresponding to efficiency of the exercise based on the piece of the first data and the piece of the second data.

11. A non-transitory computer-readable storage medium having stored thereon a program executable by a computer which comprises a controller, the program controlling the controller to perform functions comprising:
acquiring acceleration of a user as the user performs an exercise;
acquiring a piece of a first data corresponding to a total amount of mechanical work of the exercise for a predetermined time, based on the acceleration;
acquiring a piece of a second data corresponding to one of velocity and kinetic energy in a certain direction among directions related to the exercise for the predetermined time, based on the acceleration; and
acquiring a piece of a third data corresponding to efficiency of the exercise based on the piece of the first data and the piece of the second data.
